(19) United States
(12) Patent Application Publication
(10) Pub. No.: US 2022/0273491 A1
(43) Pub. Date: Sep. 1, 2022

(54) CONTROL DEVICES AND METHODS

(71) Applicant: CAMBRIDGE MECHATRONICS LIMITED, Cambridge, Cambridgeshire (GB)

(72) Inventor: Andrew Benjamin Simpson Brown, Cambridge (GB)

(21) Appl. No.: 17/634,452

(22) PCT Filed: Aug. 14, 2020
(86) PCT No.: PCT/GB2020/051960

§ 371 Date: Feb. 10, 2022

(30) Foreign Application Priority Data
Aug. 14, 2019 (GB) 1911646.6

(51) Int. Cl. A61F 9/007; H01H 61/01

(52) U.S. Cl. CPC A61F 9/00781; H01H 61/0107; A61F 2210/0023; A61F 2250/0001

(57) ABSTRACT

A flow control device (2) having: an outer wall; a static part (10) enclosed by the outer wall and at least partially defining a fluid path (42); a movable element which is movable relative to the static part (10) and arranged such that movement of the movable element relative to the static part (10) causes the fluidic resistance of the fluid path (42) to change; and an actuator arrangement (30″) arranged such that when energy is supplied to the actuator arrangement it causes the movable element to move relative to the static part, wherein the actuator arrangement (30″) and/or movable element are arranged such that the movable element does not move relative to the static part (10) when no energy is supplied to the actuator arrangement, and further wherein the actuator arrangement (30″) and the movable element are positioned within the fluid path (10).

The present application generally relates to control devices and methods, and particularly, but not exclusively, to flow control devices and methods, more particularly those in which the rate of flow is controlled by a heat-activated actuator.

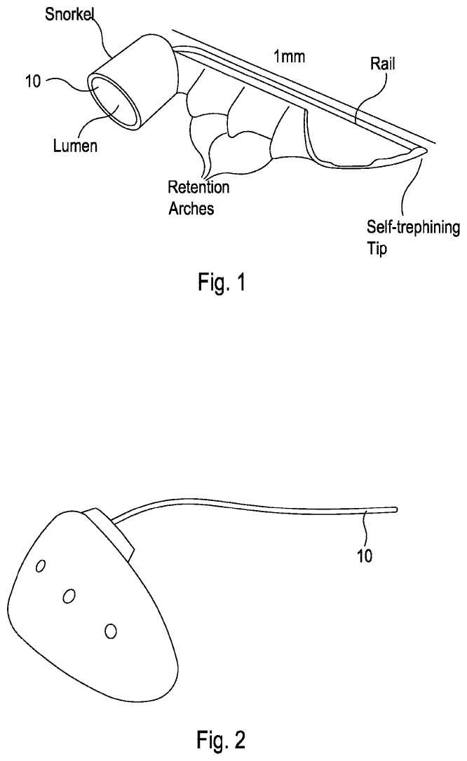

A typical stent for use with a glaucoma implant is shown in FIG. 1 and a typical shunt for use with a glaucoma implant is shown in FIG. 2. Both of these devices have a tube 10 that allows fluid to flow out of the eye. In typical implants, the rate of flow of the fluid is defined by the fixed configuration of the implant (e.g. the cross-sectional area of the tube and/or any restriction of that tube).

However, sometimes the flow rate of fluid through the implant is found to be incorrect (either too high or too low) some time after the device has been implanted. As the device is already implanted, changing the fluid flow rate is very difficult without further surgery or implanting a new device.

Whilst stents and other medical implants are examples of devices where improved flow control is desirable, the present invention finds application in a wide range of devices in applications outside of the medical field.

WO 2019/018807 discloses adjustable flow glaucoma shunts in which a control device with adjustable flow resistance is provided.

An object of the present application is to provide control devices, in particular flow control devices, which can be remotely controlled and/or adjusted and/or activated and methods of control which can remotely control or adjust a device.

A further object of some of the arrangements in the present application is to provide control devices, in particular flow control devices, which are implantable and which can be remotely controlled and/or adjusted and/or activated without surgical intervention and methods of control of implantable devices which do not require surgical intervention.

A first approach of the present application provides a flow control device having: an outer wall; a static part enclosed by the outer wall and at least partially defining a fluid path; a movable element which is movable relative to the static part and arranged such that movement of the movable element relative to the static part causes the fluidic resistance of the fluid path to change; and an actuator arrangement arranged such that when energy is supplied to the actuator arrangement it causes the movable element to move relative to the static part, wherein the actuator arrangement and/or movable element are arranged such that the movable element does not move relative to the static part when no energy is supplied to the actuator arrangement, and further wherein the actuator arrangement and the movable element are positioned within the fluid path.

The actuator assembly and/or movable element are arranged such that the movable element does not move relative to the static part when no energy is supplied to the actuator arrangement. This allows the flow control device to be adjusted by the supply of energy to the actuator arrangement and then remain in the position to which it has been adjusted without further intervention.

This may be achieved, for example, through the intrinsic hysteretic properties of the actuator arrangement in combination with a stable thermal environment in the region of use of the device. In particular, hysteresis in the contraction/stress of actuator(s) in the actuator arrangement with respect to variation in temperature of the actuator(s) can be used to achieve this functionality.

Alternatively or additionally, the device could be manufactured with a deliberate degree of friction between the movable element and the static part which could provide a stable zero-power hold state.

Positioning the operative parts of the device so that they are enclosed by the outer wall can provide for a number of advantages. For example by having the moving parts of the device enclosed by the outer wall may be more acceptable for implantable device as the moving parts are not likely to interact with surrounding tissue (particularly when moving).

Positioning the actuator arrangement and the movable element within the flow path can allow for good control of the fluidic resistance of the fluid path through the device. This may mean that control of the fluidic resistance can be achieved with a relatively small absolute range of motion provided by the actuator arrangement.

It will be appreciated that providing the actuator arrangement and the movable element within the fluid path may include these elements being provided, at least in certain configurations, such that the fluid in the fluid path passes them rather than necessarily interacting with them. For example, the actuator arrangement and the movable element may be provided such that the fluid path surrounds them in such configurations, and/or such that they are positioned on one or more sides of the fluid path, and/or in part or whole positioned adjacent to the fluid path, albeit still in an area which is in fluidic communication with the main fluid path. This may be particularly the case when the actuator arrangement and the movable element are in a “retracted” or “open” configuration which is intended to provide for low fluidic resistance through the flow control device.

The actuator arrangement may be an assembly having one or more actuators.

The static part may include an aperture and the movable element may be a closure member which is arranged to obstruct differing proportions of the aperture dependent on the position of the closure member.

The aperture may be shaped such that incremental movement of the closure member by a certain amount causes a different absolute change in the obstructed area of the aperture depending on the position of the closure member. For example, the aperture may have a “tear drop” shape. This can allow finer control of the fluid flow through the aperture at various points in the range of movement of the closure member (i.e. the points at which the aperture is narrowest, such that an incremental movement of the closure member by a certain lateral distance results in a lower absolute change in the area of the aperture which is obstructed). Similarly, the parts of the aperture which are wider mean that, for an incremental movement of the closure member by a certain lateral distance, a greater absolute change in the area of the aperture which is obstructed results. The former feature may be desirable in positions where substantially all of the apertures are obstructed by the closure member, such that the relative effect of the change in obstructed area is much greater. Conversely the latter feature may be desirable in positions where substantially all of the apertures are unobstructed, such that a greater change in the obstructed area is required to achieve a particular relative change. In particular embodiments, the aperture may be shaped such that the relative change in the obstructed area of the aperture for a given lateral movement of the closure member is substantially the same at all (or most) positions.

In certain arrangements, the movable element is formed by the actuator assembly itself and actuation and/or relaxation of the actuator assembly may case change in the fluidic resistance of the fluid path.

For example, the actuator assembly may include an actuator having a plurality of portions (such as, for example, helical coils) and actuation of the actuator assembly may cause the plurality of portions to move closer to each other, or to move apart from each other. When the portions move closer to each other, they may obstruct more of the fluid path, and relaxation may cause the coils to separate, thus presenting less obstruction to the fluid path.

In certain embodiments the actuator arrangement is arranged such that energy can be supplied to the actuator arrangement by a laser to cause the movable element to move relative to the static part.

In certain embodiments the actuator arrangement is arranged such that energy can be supplied to the actuator arrangement by an electrical current to cause the movable element to move relative to the static part.

In certain embodiments the actuator arrangement is arranged such that energy can be supplied to the actuator arrangement by a thermal source to cause the movable element to move relative to the static part.

The actuator arrangement may include first and second actuators connected to the movable element and arranged such that when energy is supplied to the first actuator it causes the movable element to move in a first direction and when energy is supplied to the second actuator it causes the movable element to move relative to the static part in a second direction which is opposite to said first direction.

It will be appreciated that, the directions referred to in all of the approaches described herein may be linear directions, but may also be, or include, rotation in a particular sense, such that the movement in the first direction may be rotation in a first sense, in which case the movement in the second direction may be rotation in a second sense.

The flow control device may comprise a first energy-receiving region, which may be a common energy-receiving region coupled, for example thermally or electrically to, or including, the first and second actuators.

The first and second actuators may be asymmetric such that when energy is equally supplied to both of the first and second actuators, the actuators cause the movable portion to move relative to the static portion in a first direction and when energy is preferentially supplied to the second actuator, the actuators cause the movable element to move relative to the static part in a second direction which is opposite to said first direction.

It will be understood that equally supplying energy to the first and second actuators includes arrangements in which substantially the same amount of energy is supplied to each actuator, and in particular arrangements in which there are only minor variations, for example due to manufacturing tolerances or the location of the device, which have no substantive effect on the operation of the device.

For example, the device may be configured such that energy can be equally supplied to both actuators via the first energy-receiving region or can be preferentially supplied to the one actuator via the first energy-receiving region.

In certain embodiments the first energy-receiving region is thermally coupled to the actuators such that, when energy is supplied to the first energy-receiving region, a first actuator increases in temperature more quickly than a second actuator. This allows for preferential actuation of the first actuator compared to the second.

In certain arrangements, the flow control device may include, or define, a thermal path from the first energy-receiving region to the second actuator via the first actuator.

In certain embodiments, the application of energy that causes motion of the movable portion in the first direction may be characterised by: the rate at which the energy is supplied; the time period over which the energy is supplied; the total amount of energy supplied; and/or the time-profile of the rate of energy supplied.

In certain embodiments the first and second actuators may have different material properties such that they are actuated at different temperatures.

The first and second actuators may be thermally coupled to, and/or preferably coated in, different materials which preferentially absorb radiation of different frequencies such that energy can be preferentially supplied to the first or second actuator depending on a frequency characteristic of the radiation.

The first and second actuators may be connected to different electrical circuits having different resonant frequencies such that energy can be preferentially supplied to the first or second actuator by inductively coupling to the electrical circuits at different frequencies.

The flow control device may be configured such that radiation with different frequencies (from the laser source or from the induction power source) can be supplied to the first energy-receiving region to preferentially supply energy to the first or second actuator.

The first and second actuators may have different mechanical properties such that they apply different forces to the moving portion when heated.

In certain embodiments the static part is elongate and the fluid path is defined axially along at least a part of the longitudinal extent of the static part, an aperture is formed in the static part; and the movable element is arranged to move longitudinally relative to the static part so as to obstruct different proportions of said aperture.

In certain embodiments the static part is elongate and the fluid path is defined axially along at least a part of the longitudinal extent of the static part, an aperture is formed in the static part, and the movable element is arranged to move rotationally about the longitudinal axis of the static part so as to obstruct different proportions of said aperture.

In certain embodiments, actuation of the actuator arrangement causes a change in configuration of the movable portion in the fluid path such that the movable portion obstructs a different amount of a cross-sectional area of the fluid path.

In certain embodiments the movable element at least partially defines the fluid path and the movable element and/or actuator arrangement are arranged such that, when energy is supplied to the actuator arrangement, the movable element changes the size and/or shape of the fluid path.

In certain embodiments the movable element includes an obstruction element which is deployable in the fluid path and the movable element and/or actuator arrangement are arranged such that, when energy is supplied to the actuator arrangement the position of the obstruction element is changed.

A further approach of the present application provides an actuation apparatus having: a static part; a movable element which is movable relative to the static part; an actuator arrangement including first and second actuators connected to the movable element; and at least one energy-receiving region; wherein the actuator arrangement is arranged such that: when energy is supplied to the actuator arrangement it causes actuation of at least one of the first and second actuators thereby causing the movable element to move relative to the static part in a first direction associated with actuation of the first actuator or in a second direction associated with actuation of the second actuator, and when no energy is supplied to the actuator arrangement the movable element does not move relative to the static part, further wherein the at least one energy-receiving region includes a first energy-receiving region coupled to, or including, both of the first and second actuators and wherein the actuation apparatus is configured such that energy can be supplied to the first energy-receiving region so as to cause the movable element to move relative to the static part in at least one of the first direction or sense and the second direction or sense.

The actuation apparatus may be configured such that energy can be supplied to the first energy-receiving region so as to cause the movable element to move relative to the static part in either one of the second direction or sense and the second direction or sense.

The actuation apparatus of this approach may include some or all of the optional and preferred features of the first approach set out above.

In particular, the actuation apparatus of this approach may be configured to control fluid flow and/or to move part of an organ in the body (for example to open or close a blood vessel or an incision).

A further approach of the present application provides an implantable medical device comprising a flow control device according to the above described first approach or an actuation apparatus according to the above described approach, including some, all or none of the optional or preferred features of those approaches.

In some embodiments, the static part may be a main body of the device and the device may further comprise: an aperture in the main body and an auxiliary aperture at the main body; and a flow channel external to the main body for effecting fluid communication between the aperture and the auxiliary aperture. The device may further comprise a partition within the main body configured to fluidly separate the aperture and the auxiliary aperture. In use, a fluid in the main body may flow through the external flow channel by the aperture, and subsequently re-enters the main body by the auxiliary aperture. The main body may comprise an inlet and an outlet substantially opposite to the inlet at the main body. For example, the aperture and the auxiliary aperture may be positioned in the main body between the inlet and the outlet, whereby the movement of the control member relative to the aperture may vary the flow rate of a fluid flowing therebetween. Advantageously, such arrangement may allow the device to be installed in or retrofitted to a tube of a glaucoma implant, as such minimizing the amount of structural modification to be made to an existing device.

Optionally, the movable element is movable relative to the auxiliary aperture between a plurality of positions in each of which the movable element obstructs a different proportion of the auxiliary aperture. Advantageously, such arrangement may provide additional flow rate control. Alternatively, a movable element may be provided for each of the aperture and the auxiliary aperture, such that their respective obstructed areas may be independently varied.

Optionally, the flow channel comprises an annular flow channel surrounding the exterior of the main body. The flow channel may comprise an annular flow channel sealingly surrounding the exterior of the main body. For example, the annular flow channel may surround a portion of the main body and may enclose the aperture and auxiliary aperture. The annular flow channel may be in the form a sleeve. Advantageously, the annular flow channel may allow a more compacted device to be produced.

In relation to all of the above-described approaches, a number of methods may be used to provide for differential actuation of a plurality of actuators in the device. For example, in some embodiments the first actuator and the second actuator are each arranged to couple inductively to alternating fields at different frequencies from each other.

In other embodiments the first actuator and the second actuator each have coatings, the coatings being selected to absorb incident radiation at different frequencies from each other.

The actuator assembly, or individual actuators in the actuator assembly, in the above-described approaches may be formed integrally with the movable element. This reduces the number of connections and therefore potential failure points as the movable element and actuator assembly can be made from a single piece of material. However, it also requires the movable element to be at least partly made from the actuator material which may not always be acceptable or desirable.

The movable element may include a seal, wherein at least part of the seal contacts the static part at least during part of the motion of the movable element. The seal may prevent passage of fluid between the movable element and the static part and thus allow for more precise control of the direction and amount of flow through the device. The seal may be integrally formed with the rest of the movable element or attached thereto. In some arrangements a seal is not necessary where the movable element is manufactured to high tolerances to be a snug fit to the static part.

A number of alternative materials exist which may be used for the actuator(s) in the device. For example, the actuator(s) may be formed from a shape-memory alloy, a physically crosslinked shape memory polymer or a chemically crosslinked shape memory polymer.

In certain embodiments, the device is an implantable medical device or forms part of an implantable medical device. For example, the device may be, or may form part of, a stent or a shunt for use as or in conjunction with an implant for glaucoma.

The device can thus allow the flow rate through the implantable device to be adjusted some time after the operation in a non-invasive procedure.

For implantable devices, the choice of transition temperature of the actuator(s) can be important. The actuator(s) may have a transition temperature above body temperature such that the tension in the system when it is not heated will be low. Alternatively, the actuator(s) may have a transition temperature below body temperature.

A further approach of the present application provides a device having: a main body having a fluid path defined therein and an aperture providing for fluid communication between the fluid path and the exterior of the main body; a closure member arranged to be movable relative to said aperture between a plurality of positions in each of which the closure member obstructs a different proportion of the aperture; and a first actuator connected between the closure member and the body, such that actuation and/or relaxation of the actuator causes the closure member to move between said plurality of positions, wherein the actuator is formed from a heat-activated material. For example, the aperture may provide for fluid communication from the fluid path to the exterior of the main body, or the aperture may provide for fluid communication from the exterior of the main body to the fluid path. The device can permit the flow rate of fluid from the fluid path to the exterior of the main body (or vice-versa) to be controlled by heat-actuation of the actuator. Such actuation can be done remotely. For example, where the device is an implantable device, heat can be transferred to the actuator after the device has been implanted and without further surgical intervention. The operation of the device is also reversible, for example by relaxation or actuation of the actuator so as to cause the closure member to move in the opposite direction.

The devices or apparatuses of the above approaches may include any combination of some, all or none of the above-described preferred and optional features.

A further approach of the present application provides a method of controlling an actuation apparatus, the actuation apparatus having a static part and a movable element movable relative to the static part, and an actuator arrangement, the actuator arrangement having first and second actuators connected to the movable element, the method including the step of either: supplying energy to the first actuator thereby causing the first actuator to exert a force on the movable element and to move relative to the static part in a first direction, or supplying energy to the second actuator thereby causing the second actuator to exert a force on the movable element and to move the movable element relative to the static part in a second direction which is opposite to said first direction, wherein energy to cause the movable element to move relative to the static part in one of the first and second directions is supplied via a first energy-receiving region coupled to, or including, both of the first and second actuators, further wherein the valve is arranged such that the movable element does not move relative to the static part when no energy is supplied to both the first actuator and the second actuator.

Energy to cause the movable element to move relative to the static part in the other direction may also be supplied via the first energy-receiving region.

In certain embodiments, the first and second actuators are formed from heat-activated material, the steps of supplying energy including either: inductively coupling to the first actuator at a first predetermined frequency so as to induce a current flow in the first actuator, or inductively coupling to the second actuator at a second predetermined frequency, which is different from said first predetermined frequency, so as to induce a current flow in the second actuator.

In certain embodiments the first and second actuators are formed from heat-activated material, the steps of supplying energy including either: irradiating the device with radiation at a first predetermined frequency, which radiation is absorbed by the first actuator to a greater extent than it is absorbed by the second actuator, so as to heat the first actuator relative to the second actuator, or irradiating the device with radiation at a second predetermined frequency, which is different from said first predetermined frequency, and which radiation is absorbed by the second actuator to a greater extent than it is absorbed by the first actuator, so as to heat the second actuator relative to the first actuator.

In certain embodiments the first and second actuators are formed from heat-activated material, the steps of supplying energy including either: irradiating the device with radiation such that said radiation is incident on the first actuator and is not incident on the second actuator, so as to heat the first actuator relative to the second actuator, or irradiating the device with radiation such that said radiation is incident on the second actuator and is not incident on the first actuator, so as to heat the second actuator relative to the first actuator.

In certain embodiments the first and second actuators are asymmetric such that supply of energy to the flow control device as a whole results in selective actuation of either the first or the second actuator based on one or more of the following characteristics of the supplied energy: the rate at which the energy is supplied; the time period over which the energy is supplied; the total amount of energy supplied; and/or the time-profile of the rate of energy supplied.

In certain embodiments the first and second actuators have different material properties such that the first actuator has a higher actuation temperature than the second actuator and the steps of supplying energy include: actuating the first actuator by supplying a first dose of heat energy to the flow control device at a position proximal to the first actuator, the first dose delivering sufficient energy to cause actuation of the first actuator, the duration of the supply of the first dose being sufficiently short to prevent transfer of sufficient energy to the second actuator to cause actuation of the second actuator and thus causing movement of the movable element in the first direction; actuating the second actuator by supplying a second dose of heat energy to the flow control device at a position proximal to the first actuator, the second dose being of lower power and longer duration than the first dose, such that the second dose is sufficiently long for sufficient heat energy to transfer to the second actuator to cause actuation of the second actuator, but insufficient powerful to cause actuation of the first actuator, and thus causing movement of the movable element in the second direction.

In certain embodiments the first and second actuators have different mechanical properties such that, the second actuator, when actuated, exerts a greater force on the movable element than the first actuator, when actuated, and the steps of supplying energy include: actuating the first actuator by supplying a first dose of heat energy to the flow control device at a position proximal to the first actuator, the first dose delivering sufficient energy to cause actuation of the first actuator, the duration of the supply of the first dose being sufficiently short to prevent transfer of sufficient energy to the second actuator to cause actuation of the second actuator, and thus causing movement of the movable element in the first direction; actuating the second actuator by supplying a second dose of heat energy to the flow control device at a position proximal to the first actuator, the second dose being of longer duration than the first dose, such that the second dose is sufficiently long for sufficient heat energy to transfer to the second actuator to cause actuation of the second actuator, and thus causing movement of the movable element in the second direction as a result of the greater force exerted on the movable element by the second actuator compared to the force exerted by the first actuator.

In all of the above approaches, the actuation apparatus may be arranged to control the flow rate through a flow control device. This may be a flow control device according to any one of the above-described approaches, but need not be.

A further approach of the present application provides a method of adjusting the flow rate through a flow control device, the device having a flow control member and at least two actuators formed from heat-activated material coupled to the flow control member and arranged, on actuation of a respective one of said actuators, to cause the flow control member to increase or decrease the flow rate, the method including the step of either: inductively coupling to a first of said actuators at a first predetermined frequency so as to induce a current flow in said first actuator, or inductively coupling to a second of said actuators at a second predetermined frequency, which is different from said first predetermined frequency, so as to induce a current flow in said second actuator.

A further approach of the present application provides a method of adjusting the flow rate through a flow control device, the device having a flow control member and at least two actuators formed from heat-activated material coupled to the flow control member and arranged, on actuation of a respective one of said actuators, to cause the flow control member to increase or decrease the flow rate, the method including the step of either: irradiating the device with radiation at a first predetermined frequency, which radiation is absorbed by a first of said actuators to a greater extent than it is absorbed by a second of said actuators, so as to heat the first actuator relative to the second actuator, or irradiating the device at a second predetermined frequency, which is different from said first predetermined frequency, and which radiation is absorbed by said second actuator to a greater extent than it is absorbed by the first actuator, so as to heat said second actuator relative to the first actuator.

A further approach of the present application provides a method of adjusting the flow rate through a flow control device, the device having a flow control member and at least two actuators formed from heat-activated material coupled to the flow control member and arranged, on actuation of a respective one of said actuators, to cause the flow control member to increase or decrease the flow rate, the method including the step of either: irradiating the device with radiation such that said radiation is incident on a first of said actuators and is not incident on a second of said actuators, so as to heat the first actuator relative to the second actuator, or irradiating the device with radiation such that said radiation is incident on the second actuator and is not incident on the first actuator, so as to heat the second actuator relative to the first actuator.

The methods of the above approaches may be used with a device according to any of the above-described approaches, including some, all or none of the optional and preferred features of that approach.

Embodiments of the present application will now be described by way of example with reference to the accompanying drawings in which:

FIG. 1 shows a typical stent for use with a glaucoma implant and has already been described;

FIG. 2 shows a typical shunt for use with a glaucoma implant and has already been described;

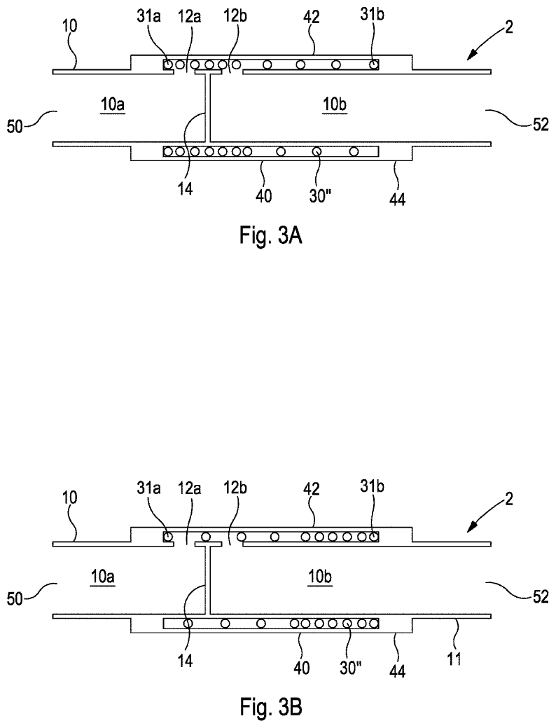

FIGS. 3A and 3B show sectional views of a device according to a further embodiment of the present application;

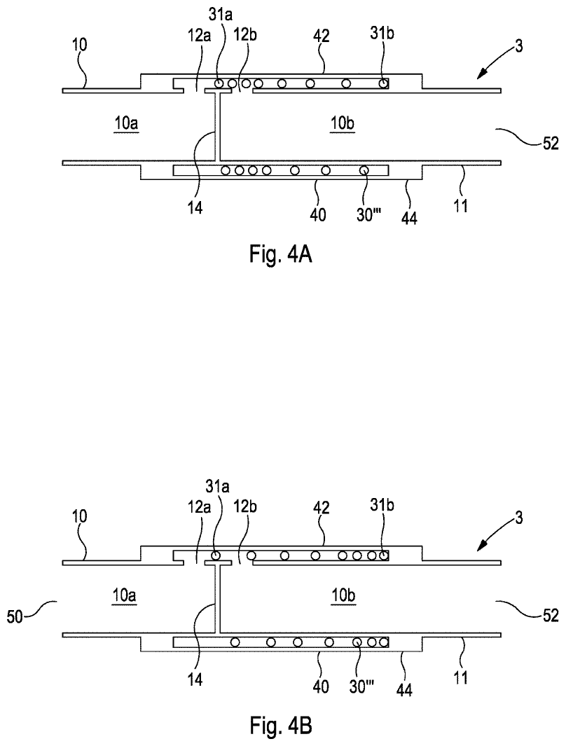

FIGS. 4A and 4B show sectional views of a device according to a further embodiment of the present application;

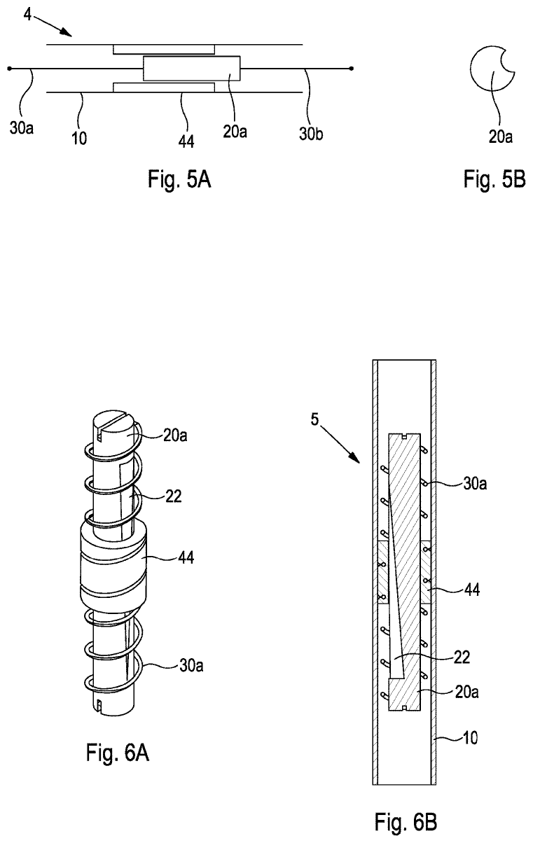

FIGS. 5A and 5B show, respectively, sectional and end views of a device according to a further embodiment of the present application;

FIGS. 6A and 6B show, respectively, perspective and cross-sectional views of a device according to a further embodiment of the present application;

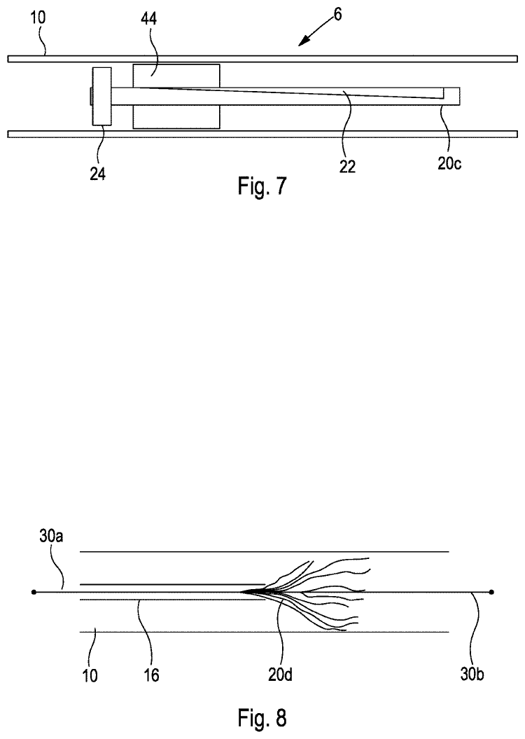

FIG. 7 shows a sectional view of a device according to a further embodiment of the present application;

FIG. 8 shows a sectional view of a device according to a further embodiment of the present application;

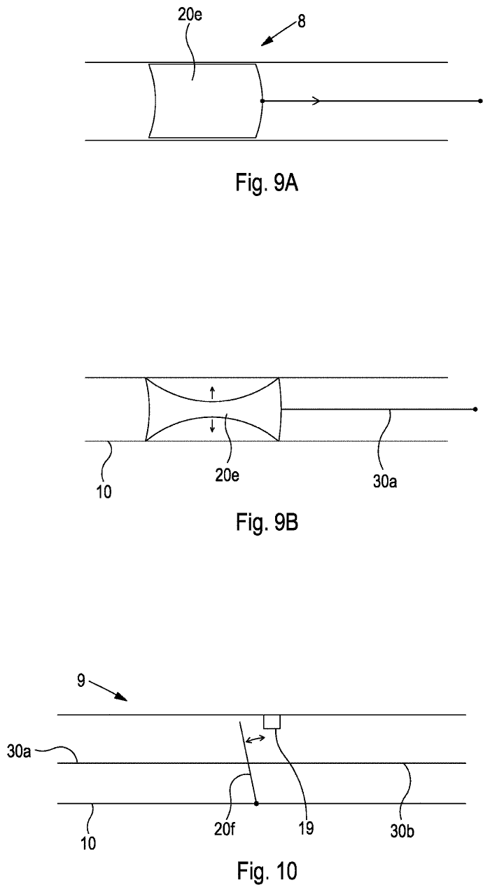

FIGS. 9A and 9B show sectional views of a device according to a further embodiment of the present application in, respectively, a relaxed and a contracted state;

FIG. 10 shows a sectional view of a device according to a further embodiment of the present application;

FIGS. 11A and 11B show, respectively, a perspective view of an element of a device according to a further embodiment of the present application, and a sectional view of the device of that embodiment;

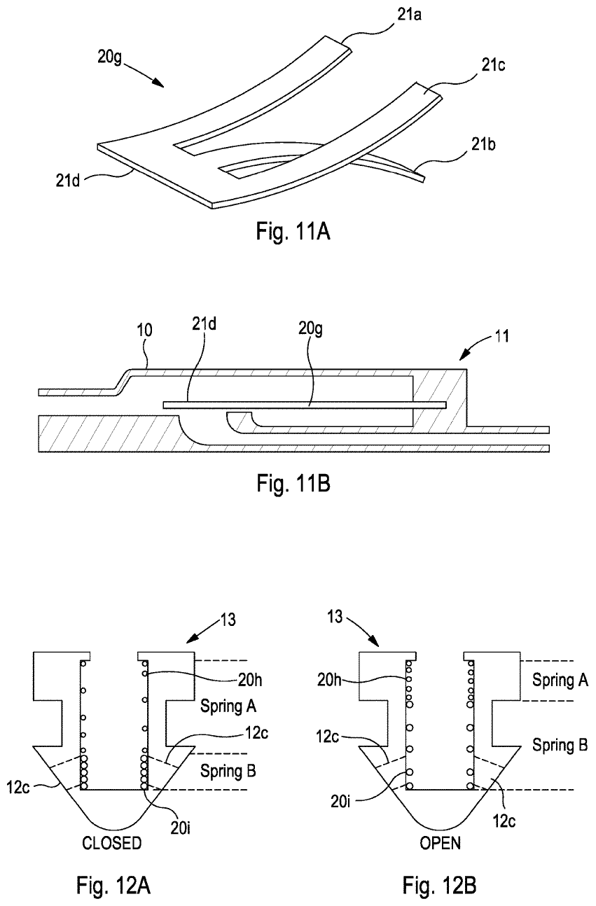

FIGS. 12A and 12B show sectional views of a device according to a further embodiment of the present application in, respectively, closed and open positions;

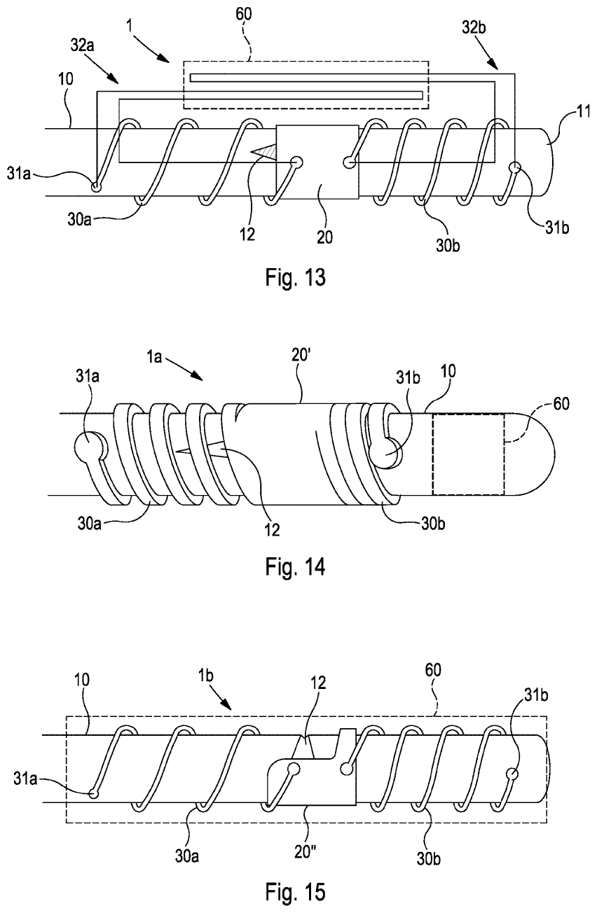

FIG. 13 shows a device according to a further embodiment of the present application;

FIG. 14 shows a device according to a further embodiment of the present application;

FIG. 15 shows a device according to a further embodiment of the present application; and

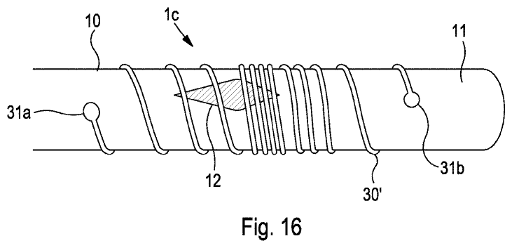

FIG. 16 shows a device according to a further embodiment of the present application.

Devices according to embodiments of the present application use heat-activated material as an actuator/actuators to control movement of components of the device. Examples of heat-activated material that may be used in these devices are:
SMA (Shape Memory Alloy); this is typically a nickel-titanium alloy (e.g. Nitinol), but may also contain tertiary components such as copper.
Physically crosslinked SMP (Shape Memory Polymer); representative shape memory polymers include polyurethanes, polyurethanes with ionic or mesogenic components made by a prepolymer method. Other block copolymers also show the shape-memory effect, including: a block copolymer of polyethylene terephthalate (PET) and polyethyleneoxide (PEO), block copolymers containing polystyrene and poly(1,4-butadiene), and an ABA triblock copolymer made from poly(2-methyl-2-oxazoline) and polytetrahydrofuran.
Chemically crosslinked SMPs; examples include crosslinked polyurethane or PEO-based crosslinked SMPs. The network polymer can be synthesized by either polymerization with multifunctional (3 or more) crosslinker or by subsequent crosslinking of a linear or branched polymer.

In devices having two or more actuators, different actuators may be made from different ones of the above materials (or from two different materials of the same type). This may be useful to achieve an arrangement in which the actuators have different properties, either in terms of their mechanical properties or how they are actuated.

Embodiments of devices according to the present application will now be described. Where similar or identical components are used in the different embodiments, they will be given the same reference numerals. For efficiency, description of similar or identical elements may not be repeated between the embodiments and characteristics and features of elements are to be understood as applying to those elements in all embodiments unless the description indicates otherwise.

FIGS. 3A and 3A show sectional views of a device 2 according to an embodiment of the present application. Similar to the device 1c as shown in FIG. 16 and described further below, the device 2 comprises a coil of SMA actuator wire 30″ (or other heat-activated actuators) formed around the exterior of the tube 10 between the anchor positions 31a, 31b. In this embodiment, the tube 10 is separated into an upstream tube portion 10a and a downstream tube portion 10b, wherein the two tube portions 10a, 10b are fluidly separated by a partition 14 extending across the tube 10. The upstream tube portion 10a and the downstream tube portion 10b are respectively in fluid communication with an inlet 50 and an outlet 52. The tube 10 comprises holes 12a, 12b each configured to fluidly communicate with the respective upstream tube portion 10a and downstream tube portion 10b. The holes 12a, 12b are identical apertures as shown in the illustrated example, but they can be apertures of different sizes and/or shapes.

As shown in FIGS. 3A and 3B, the device 2 further comprises a sleeve 40 surrounding a portion of the tube 10, thereby forming an annular flow channel 42 therebetween. Each of the end portions of the sleeve 40 comprises a sealing element 44 for sealing the annular flow channel 42. In use, a fluid flow path extends, through the annular flow channel 42, between the upstream tube portion 10a and the downstream tube portion 10b. The annular gap, or the depth, of the annular flow channel 42 may be the same, or slightly wider, than the diameter (or gauge) of the SMA actuator wire 30″. In this case, the flow path for all of the fluid, or a substantial portion of the fluid, may spirally extend along the annular flow channel 42. Hence, fluid may flow between the coils of the actuator wire 30″. In other embodiments, the annular flow channel 42 may have the same hydraulic diameter as that of the tube 10 to avoid constricting the fluid flow. Hence, fluid may flow over the surface of the SMA actuator wire 30″. In other embodiments, the annular flow channel 42 may have a larger or a smaller hydraulic diameter than that of the tube 10.

In use, the coils of the actuator 30″ act to obstruct both holes 12a, 12b. The flow rate of fluid passing through the annular flow channel 42 can be controlled by varying the area of the holes 12a, 12b that is being obstructed by the coils of the actuator 30″. This can be achieved by changing the separation between the coils in the actuator 30″ that is overlaying each of the holes 12a, 12b. For example, FIG. 3A shows the device 2 being put into a closed position, where the coils of the actuator 30″ adjacent to the holes 12a and 12b are contracted or closed up. Hence, in the closed position, the spacings between the coils adjacent to the holes 12a and 12b reduce to a minimum, or zero (e.g. the coils are in contact with each other). This causes the holes 12a, 12b to be significantly or completely obstructed by the actuator 30″, resulting in a reduced flow or blockage thereacross. On the other hand, FIG. 7B shows the device 2 being put in an opened position, where the coils of the actuator 30″ adjacent to the holes 12a and 12b are relaxed or extended. Hence, the spacings between the coils at this location increases, thereby allowing the fluid to pass through the holes 12a, 12b at a higher flow rate.

A change in the separation between actuator 30″ coils also affects the flow resistance along the annular flow channel 42, thereby providing additional degree of flow control. For example, when the device is put into the closed position as shown in FIG. 3A, the portion of annular flow channel 42 in between the two holes 12a, 12b is occupied by an increased number of actuator coils 30″ in comparison to the opened position of FIG. 3B. As a result, the fluid flow path narrows and thus resulting in a reduced fluid flow rate. Furthermore, in embodiments where the annular gap of the annular flow channel matches the gauge of SMA actuator wire 30″, a change in the separation between the coils results in narrowing or widening of the spiral fluid flow path, and therefore effecting a change in the fluid flow rate.

In the illustrated embodiment, at a given temperature, the actuator 30″ is configured to obstruct or to cover similar amount of opening in each of holes 12a, 12b. Hence, the flow resistances across the different holes 12a, 12b are substantially similar. In other embodiments, the actuator 30″ may be configured to obstruct or to cover different amount of opening in the holes 12a, 12b, and as a result the flow resistances through the different holes 12a, 12b may be different to each other.

The device 2 differs to the previous embodiments in that the overall direction of the fluid flow remains unchanged. Hence, a fluid may enter, via inlet 50, and subsequently be discharged, via outlet 52, from the tube 10 in substantially the same direction. Further embodiments according to the present application may utilise any one of the closure members 20, 20′, 20″ and corresponding SMA actuator wires 30a, 30b arrangements of FIGS. 3-5 in place of the actuator 30″ of FIGS. 3A and 3B, for controlling the degree of obstruction or coverage over holes 12a, 12b.

FIGS. 4A and 4B respectively shows a device 3 in an opened position and closed position according a further embodiment of the present application. The device 3 is structurally and functionally similar to the device 2 as shown in FIGS. 3A and 3B, apart from that the actuator 30′ in device 3 is configured to cover only one of the holes 12a, 12b. As shown in FIGS. 4A and 4B, one end of the actuator 30′″ is anchored to the tube 10 at a location 31a between the holes 12a, 12b. Such arrangement allows one of the holes 12a, 12b to remain unobstructed, and thereby reduces the flow resistance along the fluid flow path.

In the illustrated embodiment, the actuator 30′″ is configured to cover or to obstruct the hole 12b opened at the downstream tube portion 10b. In other embodiments, the actuator may be configured to cover or to obstruct the hole 12a opened at the upstream tube portion 10a.

FIGS. 5A and 5B show, respectively, a sectional view of a device 4 according to a further embodiment of the present application and a cross-sectional view of the movable element 20a of that device. In the device of this embodiment, the movable element 20a is a needle-like element that is arranged to move within a collar 44, and the cross section of the movable element 20a is constant along its length (or at least the portion of its length that will be positioned within the collar 44 at any time), such that a flow path is defined between the movable needle element 20a and the collar 44. The position of the movable element 20a controls the fluid flow rate past the collar by adjusting the length of the restricted flow path.

In FIG. 5A, the actuators 30a, 30b are linear SMA actuator wires substantially aligned along the longitudinal axis of the device 4. However, opposing coiled SMA actuator wires, such as those shown in FIGS. 6A and 6B and discussed in more detail below, could also be used.

FIGS. 6A and 6B show, respectively, a perspective view of a device 5 according to a further embodiment of the present application and a cross-sectional view from the side of the device 5. For clarity, in FIG. 6A, the outer tube 10 of the device is not shown.

Like the embodiment shown in FIGS. 5A and 5B and described above, the movable element 20b of the device 5 is a needle-like element that is arranged to move within a collar 44. However, as shown in FIG. 6B, the needle-like movable element 20b has a channel 22 of varying cross-section along the longitudinal extent of the movable element. This configuration of the movable element 20b means that the cross-sectional area of the narrowest point of the channel formed between the movable element 20b and the collar 44 varies depending on the position of the movable element 20b within the collar 44. Thus positioning of the movable element 20b can control the fluid flow through the collar 44.

A single SMA actuator wire 30a is wound around the movable element 20b and the collar 44 in a helical arrangement, passing through a defined channel on the outer part of the collar 44. In alternative arrangements, two actuator wires could be provided on either side of the collar, or a single actuator wire on one side of the collar and a biasing element (such as a coiled spring) on the other.

Needle designs such as those shown in FIGS. 5 and 6 and described above can be difficult to manufacture with sufficiently precise tolerances, in particular between the outer diameter of the movable element and the inner diameter of the collar. If the movable element is too large, then it may not move freely within the collar and may get jammed and/or be difficult to control due to large friction forces which have to be overcome by the forces exerted by the actuators. Conversely, if the needle diameter is too small relative to the inner diameter of the collar, then there may always be sufficient clearance between the components for fluid to leak through even when the device is in a supposedly “closed” state.

A first approach to addressing the above difficulty is to design both the movable element and the inside of the collar so that they have corresponding conical, or frustro-conical shapes, thereby ensuring that there is a position of the movable element in which the outer surface of the movable element is in complete contact with the inner surface of the aperture in the collar. However, in such arrangements, the position of the movable element at which full contact, and therefore sealing, occurs is not always known and will, again, depend on the manufacturing tolerance of the components.

A second approach to ensure that a full sealed position is always achievable is illustrated in the embodiment shown in FIG. 7. In the device 6 shown, a needle-type movable element 20c having a channel 22 is arranged to move within a collar 44 (for clarity, the actuator wires and other details are not shown in FIG. 7). At one end of the movable element 20c, there is an end stop 24. When the movable element 20c is moved to the furthest extent possible in one direction (to the right as shown in FIG. 7), the end stop 24 abuts the face of the collar 44 and can thereby seal against it, preventing any flow through the collar. The end stop 24 may be designed with or provided with a sealing element (such as an O-ring or similar) to assist in this sealing.

FIG. 8 shows a device 7 according to a further embodiment of the present application. An internal chamber 16 is arranged inside the tube 10. The movable element is comprised of one or more (several in the arrangement illustrated in FIG. 8) obstructing devices 20d which are housed in the chamber 16. SMA actuator wires 30a, 30b are arranged such that, on actuation of one of the actuator wires 30b, the obstructing devices 18 are deployed from the chamber 16 into the fluid path, whilst on actuation of the other of the actuator wires 30a, the obstructing devices 18 are returned to the chamber 16. Differential control of the actuator wires 30a, 30b can allow control of the number and/or extent of obstructing devices 18 that are deployed within the tube 10.

The obstructing devices 18 are arranged to restrict fluid flow through the tube 10. For example, the obstructing devices may be designed to restrict fluid flow by creating a multitude of channels with a small characteristic length, thereby increasing fluidic resistance past the obstructing devices 18 and reducing the fluid flow through the tube 10.

FIGS. 9A and 9B show a device 8 according to a further embodiment of the present application. In this device 8, the movable element is a mesh 20e which is arranged around the outside of the inner diameter of the tube 10 and is arranged to normally lie flush with the inner surface of the tube 10 as shown in FIG. 9A so that the fluid can flow through the tube 10 unimpeded. On actuation of the actuator wire 30a, the mesh 20e distorts so that it is deployed within the body of the tube 10 as shown in FIG. 9B and so obstructs the fluid flow through the tube, by reducing the cross-sectional area of the flow path through the tube 10 and/or by increasing the turbulence of the fluid flowing through the mesh 20e. Control of the extent to which the mesh obstructs the tube 10 (or conversely the size of the remaining unobstructed fluid path through the tube) can allow control of the fluid flow rate.

The mesh 20e may itself be made of a heat-activated material such as SMA. In such an arrangement, the mesh 20e may be configured such that, on activation by heating, it returns to its original shape around the edge of the inside of the tube 10 (as shown in FIG. 9A).

FIG. 10 shows a device 9 according to a further embodiment of the present application. In this device 9, the movable element is a flap 20f which can be pivoted within the inner portion of the tube 10 (for example at one edge, as shown in FIG. 14, or about a central axis) and, in a fully closed position may lie against a valve seat 19. The movement and position of the flap 20f is controlled by opposed SMA actuator wires 30a, 30b.

FIGS. 11A and 11B show, respectively, a further arrangement of a flap 20g which may be used as the movable element in a device 11 according to a further embodiment of the present application, and a cross-sectional view of the device 11.

The flap 20g of this device 11 is formed of a single sheet of SMA metal. The natural shape of the flap 20g is shown in FIG. 11A with the two outer arms 21a, 21c angling upwards from the common portion 21d, whilst the central arm 21b angles downwards. During construction of the device, the flap 20g is deformed so that the three arms 21a-21c are forced to be co-planar (horizontal in the arrangement in FIG. 11B). Then, if the central arm 21b is heated, the common portion 21d will move downwards in the arrangement shown in FIG. 15B thereby reducing or closing the fluid flow path through the device 11. Conversely, if the outer arms 21a, 21c are heated then the common portion 21d will move up, opening the fluid flow path and allowing greater fluid flow.

FIGS. 12A and 12B show a further device 13 according to an embodiment of the present application. In this device 13 the movable element is formed from two SMA compression coil springs 20h, 20i. These springs are arranged to work against each other and, as they are formed of SMA, are also the actuators of the device 13. The tube 10 is a closed-ended tube and has one or more exit passages 12c arranged adjacent the closed end.

The springs 20h, 20i are formed of wires of different diameters but are otherwise similar, having an outer diameter of 150 μm and a natural length of 150 μm when extended and 5 coils. The first spring 20h is formed from wire with a 25 μm diameter whilst the second spring 20i is formed from wire with a 35 μm diameter.

FIG. 12A shows the device 13 in a “closed” position in which the second spring 30h blocks, partially or completely, the exit passages 12c. Heating of the second spring 20i will generate a force that is able to overcome the hysteresis in the material of the first spring 20h and so allow the second spring 20i to expand and compress the first spring 20h, thus arriving at the “open” position shown in FIG. 12B.

The transition from “closed” to “open” can also be achieved by heating both springs. If both the first and second springs are heated (for example by a spread, longer length laser pulses or sequence of pulses), the larger cross-sectional area of the second spring 20i (approximate twice that of the first spring 20h) will generate a force that is able to overcome the hysteresis in the first spring 20h.

The device can then be returned, partially or completely, to the closed state by heating the first spring 20h only (for example with a focused laser pulse) so that it heats up whilst the second spring 20i remains cool. If the temperature differential between the springs is sufficiently large (for the dimensions set out in this embodiment, that difference has been found to be typically around 35° C.) then the first spring will be able to overcome the hysteresis in the second spring 20i.

The heating of the heat-activated actuator(s), such as SMA material, in order to cause the moving portion to move, could be achieved in a number of ways.

In one arrangement, the material could be heated by passing a current through it. This current might come from a local or external power supply. Alternatively, the current might be induced in the wire by inductive coupling with an external alternating field. Where there are two actuators, the two actuators might be designed so that they couple to two different frequencies of the inductive power source, thus allowing the two actuators to be heated differentially.

In another arrangement, the material could be heated by external radiation such as a visible or infra-red laser. The external radiation could be focussed so that one actuator is heated preferentially over another actuator, thus allowing differential actuation. Alternatively or additionally, different actuators, or portions of the actuators, could be treated (for example with a surface coating) so that the different actuators heat at different rates depending on the nature (e.g. the frequency) of the incident radiation.

In some implementations of the embodiments of the present application, for example when the devices is used as a flow adjuster for a glaucoma stent, it may be desirable to place the device in a position where it is not possible to access regions of the device that are close to one of the actuators.

This may means that while it is possible to heat one of the actuators to move the movable element in one sense (e.g. a first direction), it is not possible to directly heat the opposing actuator to move the movable element in the reverse fashion (e.g. the opposing direction).

Accordingly, the devices in the following embodiments of the present application can be actuated in either direction by only applying heat to one region of the device.

At a general level, this is achieved by providing actuators which have asymmetry, and preferably a significant asymmetry.

In a first such embodiment, the device is constructed such that the temperature at which the opposing actuators actuate is different.

For example, in one arrangement of such a device, the actuators consist of two opposing tension springs constructed from SMA. The transition temperature of SMA is characterised by four temperatures: Austinite start (As), Austenite finish (Af), Martensite start (Ms) and Martensite finish (Mf). The device is assumed to be normally at a temperature of 36° C.

A first of the springs (A) is made of a material that has an As temperature of 45° C. and a second of the springs (B) is made of a material with an As temperature of 60° C. The device is constructed so that both springs are extended from their natural length (length at temperatures greater than Af for each material). The device is also constructed so that the location of heating is near to spring B, but further from spring A.

To actuate the device in the first direction a short pulse of heat is applied to the heating location. This short pulse heats spring B, but the pulse is sufficiently short that the heat dissipates before it is able to significantly heat spring A. This causes spring B to contract towards its natural length, moving the moving portion in a first direction.

To actuate the device in the second direction (opposite to the first direction) a longer, lower power pulse of heat is applied to the heating location. This long pulse heats both spring A and spring B since the duration of the pulse is long enough to allow the heat to propagate from the heating location to both springs. However, the low power of the pulse is not sufficient to heat spring B above As, but is sufficient to heat spring A above its As since the As of spring A is significantly lower than the As of spring B. This causes spring A to contract towards its natural length, moving the moving portion in the second direction.

In a second such embodiment, the device is constructed with a difference in effective cross-sectional area between the actuators. The effective cross-sectional area in each of the actuators may be substantially different. For the present purposes the effective cross-sectional area of the actuators is defined as the relationship between the force applied to an actuator in a direction opposite to its actuation direction and a measure of the stress in that element, where a larger cross-sectional area means a lower stress for a given force.

In one arrangement of a device according to this embodiment the actuators consist of a single SMA tension spring that pulls the moving portion a first direction and a pair of SMA tension springs that pull the moving portion in a second direction (opposite to the first direction).

The device is constructed so that the location of heating is near to the lone spring, but further from the pair of springs.

To actuate the device in the first direction a short pulse of heat is applied to the heating location. This short pulse heats the lone spring, but the pulse is sufficiently short that the heat dissipates before it is able to significantly heat the pair of springs. This causes the lone spring to contract towards its natural length, moving the moving portion in a first direction.

To actuate the device in the second direction (opposite to the first direction) a longer, lower power pulse of heat is applied to the heating location. This long pulse heats both the lone spring and the pair of springs since the duration of the pulse is long enough to allow the heat to propagate from the heating location to all the springs. When heated all the springs try to contract, but since two springs re pulling in the second direction while only one spring is pulling in the first direction the pair of springs prevail and they contract towards their natural length, moving the moving portion in the second direction.

Similarly instead of using a pair of springs, a spring of the same diameter made with a thicker wire could be used, or a spring with the same wire diameter, but a smaller coil diameter could also be used.

The material used for the actuators can be selected so that the transition temperature of the material has a particular relationship with the environment in which the device is going to be used (e.g. body temperature in the case of implantable devices).

In the case where the transition temperature of the actuator material is above the temperature of the environment, then the tension in the system when it is not heated will be low.

In the case where the transition temperature of the actuator material is below the temperature of the environment, then the material will behave super elasticity, and so the system will be under tension.

In each case the zero hold power requirement could be achieved via the hysteresis of the thermally active material or through friction deliberately added to or incorporated in the system.

In certain arrangements the fully open and fully closed positions of the moving portion may be at points where the thermally activated material is not 100% of the way through the thermal transition. This is because there may be some relaxation of the material despite the hysteretic behaviour that needs to be accounted for.

FIGS. 13-16 show devices 1 according to further embodiments of the present invention which illustrate specific configurations for the supply of energy to the actuators.

FIG. 13 shows a device 1 according to a further embodiment of the present application. The device is formed of a static tube 10 which is closed at one end 11 and a hole 12 is formed in the side of the tube. Regulation of the size of that hole is used to control fluid flow through and out of the fluid path (not shown) inside the tube 10 and onwards.

A movable element 20, which in this embodiment is a cylinder with an interior diameter that is slightly larger than the exterior diameter of the tube is positioned around the outside of the tube 10. The movable element 20 can move longitudinally along the tube and the position of the movable element 20 relative to the hole 12 alters the amount of the hole that is covered.

The movable element 20 is connected to two lengths of SMA actuator wire 30a, 30b that are wound around the tube and connect to the exterior of the tube at anchor positions 31a, 31b which are removed from the position of the hole 12.

The actuator wires 30a, 30b are electrically connected to an energy-receiving area 60 by conductive elements 32a and 32b respectively. Conductive elements 32a, 32b may be wires or other conductors (such as printed circuit board tracks). Energy-receiving area 60 is composed of at least one coils of each of the conductive elements 32a, 32b and is thus arranged to inductively couple energy from an external power source to a respective one of the actuator wires 30a, 30b. The coils are arranged so that it is possible to selectively couple to the conductive elements 32a, 32b and thus supply energy to the individual actuator wires 30a, 30b.

Whilst the energy-receiving area 60 is shown adjacent to the device 1 in FIG. 13, it will be appreciated that, provided that electrical connection is provided through conductive elements 32a, 32b, the energy-receiving area can be provided in other locations and, in particular, may be remote from the movable element 20 and actuator wires 30a, 30b, thus meaning that the device itself can be implanted in a position where inductive couple directly to the device would not be possible but power can be supplied through the energy-receiving area 60, which can be positioned in a location where coupling is possible and/or is easier/more efficient.

When one of the lengths of SMA actuator wire 30a, 30b is heated above the temperature of the other wire, this causes the heated actuator wire to contract, exerting a force on the movable element in the direction of the respective anchor position of the heated wire. The differential force on the movable element 20 causes it to move along the length of the tube in the direction of the anchor position of the heated wire, thus altering the amount of the hole 12 that is obscured by the movable element and thus altering the fluidic resistance of the hole and thus the flow rate through the tube 10 as a whole.

The hole 12 in the tube could be circular, but in this example the hole is tear drop-shaped. This can allow finer control of the fluid flow when the hole is almost completely covered as the absolute change in size of the open portion of the hole for a given lateral movement of the movable element 20 can be much less at one extreme of the motion (e.g. when the hole is almost completely obscured) than at the other end. Selection of the shape of the hole 12 can be done to provide a range of possible profiles for the relationship between the degree of motion of the movable element 20 and the effect on the fluid flow rate through the hole 12.

FIG. 14 shows a device 1a according to a further embodiment of the present application. The device 1a of this embodiment differs from that of the first embodiment above in that the movable element 20′ is made from the same material as the actuators 30a, 30b, and indeed may be formed from the same original blank of that material. The movable element 20′ is cut into a helical shape at the two extremities to form the actuators 30a, 30b and the whole is made from a shape memory alloy such as nitinol.

This design requires fewer joins than that of the previous embodiment and the cross section of the helical portion can be more easily made non-circular which can allow the stiffness of a section of the helical portion to have a higher bending moment along the length of the tube 10 than it does radially.

FIG. 14 also differs from the device shown in FIG. 13 in that the energy-receiving area 60 is located on the tube 10 at one end of the device 1a, such that it is relatively proximal to one of the actuator wires 30b, and relatively distal to the other actuator wire 30a. This arrangement of the energy-receiving area allows heat energy to be supplied to this common energy-receiving area (for example using a laser), but to differentially heat the actuator wires 30a, 30b due to their different relative positions.

In this arrangement the tube 10 may be made from a material that has high heat conductivity to allow for efficient transfer of heat energy from the energy-receiving area to the actuator wires 30a, 30b. Alternatively or additionally, heat-conductive elements or contacts may be provided to specifically facilitate this heat transfer. The use of a common heating zone to control the device can allow for preferential actuation of the two actuator wires based on differences in heating profile (time, intensity, etc.) as already discussed above.

FIG. 15 shows a device 1b according to a further embodiment of the present application. The device 1b of this embodiment differs from the devices of FIGS. 13 and 14 in that the movable element 20″ is arranged to rotate circumferentially around the tube 10 in order to change the amount of the hole 12 that is obstructed, rather than translating along the tube 10. The hole 12 in this embodiment is configured accordingly.

The device 1b in FIG. 15 is further differentiated from the devices shown in FIGS. 13 and 14 by the energy-receiving area 60 encompassing all (or substantively all) of the area in which the actuator wires 30a, 30b are located. In the device 1b of FIG. 15, the two actuator wires are manufactured from, or coated in, different materials which have different radiation absorbing properties. For example, a first actuator wire 30a may be coated in or manufactured from a first material which has a defined absorption spectrum, whilst the second actuator wire 30b may be coated in or manufactured from a second material which also has a defined absorption spectrum which has a low degree of overlap (or none at all) with the absorption spectrum of the first actuator wire.

Thus application of radiation of a particular frequency, or a particular frequency spectrum, to the energy-receiving zone 60 as a whole will result in absorption of the radiation by one of the wires preferentially to the other wire.

FIG. 16 shows a device 1c according to a further embodiment of the present application. The device 1c of this embodiment primarily differs from the devices of the embodiments shown in FIGS. 13 to 15 in that there is no separate movable element. In the device 1c, a coil of SMA wire 30′ (or other heat-activated actuator) is formed around the exterior of the tube 10 between the anchor positions 31a, 31b. The coils of the actuator 30′ act to obstruct the hole 12. The amount of the hole 12 that is obstructed can be varied by changing the separation between the coils in the portion of the actuator 30′ that is overlaying the hole 12.

For example, in one arrangement, the actuator 30′ can be formed so that, when it is heated, the coils in the middle of the actuator 30′ close up, thus obstructing more of the hole 12. In this arrangement the actuator 30′ could be formed with the coils spaced apart and the material heat-treated to set this shape. The actuator 30′ could then be reverse-wound around the tube 10 so that when the material is heated the coils contact with each other before the material of the actuator 30′ becomes fully austenite.

In an alternative arrangement, the actuator 30′ can be formed so that, when it is heated, the coils in the middle of the actuator 30′ move apart, thus obstructing less of the hole 12.

Those skilled in the art will appreciate that while the foregoing has described what is considered to be the best mode and where appropriate other modes of performing present application, the present application should not be limited to the specific configurations and methods disclosed in this description of the preferred embodiment. Those skilled in the art will recognise that present application have a broad range of applications, and that the embodiments may take a wide range of modifications without departing from any inventive concept as defined in the appended claims.

1. A flow control device having:
an outer wall;
a static part enclosed by the outer wall and at least partially defining a fluid path;
a movable element which is movable relative to the static part and arranged such that movement of the movable element relative to the static part causes the fluidic resistance of the fluid path to change; and
an actuator arrangement arranged such that when energy is supplied to the actuator arrangement it causes the movable element to move relative to the static part,
wherein the actuator arrangement and/or movable element are arranged such that the movable element does not move relative to the static part when no energy is supplied to the actuator arrangement, and
further wherein the actuator arrangement and the movable element are positioned within the fluid path.

2. A flow control device according to claim 1 wherein the movable element does not move relative to the static part when no energy is supplied to the actuator arrangement due to friction between the movable element and the static part; and/or
wherein the movable element does not move relative to the static part when no energy is supplied to the actuator arrangement due to hysteretic properties of the actuator arrangement; and/or
wherein the static part includes an aperture and the movable element is a closure member which is arranged to obstruct differing proportions of the aperture dependent on the position of the closure member; and/or
wherein the actuator arrangement is arranged such that energy can be supplied to the actuator arrangement by a laser to cause the movable element to move relative to the static part; and/or
wherein the actuator arrangement is arranged such that energy can be supplied to the actuator arrangement by an electrical current to cause the movable element to move relative to the static part; and/or
wherein the actuator arrangement is arranged such that energy can be supplied to the actuator arrangement by a thermal source to cause the movable element to move relative to the static part.

3-7. (canceled)

8. A flow control device according to claim 1 wherein the actuator arrangement includes first and second actuators connected to the movable element and arranged such that when energy is supplied to the first actuator it causes the movable element to move in a first direction and when energy is supplied to the second actuator it causes the movable element to move relative to the static part in a second direction which is opposite to said first direction.

9. A flow control device according to claim 8 comprising a first energy-receiving region coupled to, or including, the first and second actuators; and/or
wherein the first and second actuators are asymmetric such that when energy is equally supplied to both of the first and second actuators, the actuators cause the movable element to move relative to the static element in a first direction and when energy is preferentially supplied to the second actuator, the actuators cause the movable element to move relative to the static part in a second direction which is opposite to said first direction.

10-17. (canceled)

18. A flow control device according to claim 1 wherein the static part is elongate and the fluid path is defined axially along at least a part of the longitudinal extent of the static part, an aperture is formed in the static part; and the movable element is arranged to move longitudinally relative to the static part so as to obstruct different proportions of said aperture; and/or
wherein the static part is elongate and the fluid path is defined axially along at least a part of the longitudinal extent of the static part, an aperture is formed in the static part, and the movable element is arranged to move rotationally about the longitudinal axis of the static part so as to obstruct different proportions of said aperture; and/or
wherein actuation of the actuator arrangement causes a change in configuration of the movable element in the fluid path such that the movable element obstructs a different amount of a cross-sectional area of the fluid path; and/or
wherein the movable element at least partially defines the fluid path and the movable element and/or actuator arrangement are arranged such that, when energy is supplied to the actuator arrangement, the movable element changes the size and/or shape of the fluid path; and/or
wherein the movable element includes an obstruction element which is deployable in the fluid path and the movable element and/or actuator arrangement are arranged such that, when energy is supplied to the actuator arrangement the position of the obstruction element is changed.

19-22. (canceled)

23. An actuation apparatus having:
a static part;
a movable element which is movable relative to the static part;
an actuator arrangement including first and second actuators connected to the movable element; and
at least one energy-receiving region;
wherein the actuator arrangement is arranged such that:
when energy is supplied to the actuator arrangement it causes actuation of at least one of the first and second actuators thereby causing the movable element to move relative to the static part in a first direction associated with actuation of the first actuator or in a second direction associated with actuation of the second actuator, and
when no energy is supplied to the actuator arrangement the movable element does not move relative to the static part,
further wherein the at least one energy-receiving region includes a first energy-receiving region coupled to, or including, both of the first and second actuators and wherein the actuation apparatus is configured such that energy can be supplied to the first energy-receiving region so as to cause the movable element to move relative to the static part in at least one of the first direction or sense and the second direction or sense.

24. An actuation apparatus according to claim 23 configured such that energy can be supplied to the first energy-receiving region so as to cause the movable element to move relative to the static part in either one of the second direction or sense and the second direction or sense; and/or:
wherein the first and second actuators are asymmetric such that when energy is equally supplied to both of the first and second actuators, the actuators cause the movable element to move relative to the static element in a first direction and when energy is preferentially supplied to one of the first and second actuators, the actuators cause the movable element to move relative to the static part in a second direction which is opposite to said first direction; or
configured such that energy can be equally supplied to both actuators via the first energy-receiving region or can be preferentially supplied to the one actuator via the first energy-receiving region; or
wherein the first energy-receiving region is thermally coupled to the actuators such that, when energy is supplied to the first energy-receiving region, the one actuator increases in temperature more quickly than the other actuator; or
where the application of energy that causes motion of the movable element in the first direction is characterised by: the rate at which the energy is supplied; the time period over which the energy is supplied; the total amount of energy supplied; and/or the time-profile of the rate of energy supplied; or
wherein the first and second actuators have different material properties such that they are actuated at different temperatures; or
wherein the first and second actuators are thermally coupled to, preferably coated in, different materials which preferentially absorb radiation of different frequencies such that energy can be preferentially supplied to the first or second actuator depending on a frequency characteristic of the radiation; or
wherein the first and second actuators are connected to different electrical circuits having different resonant frequencies such that energy can be preferentially supplied to the first or second actuator by inductively coupling to the electrical circuits at different frequencies.

25-31. (canceled)

32. An actuation apparatus according to claim 23 wherein the first and second actuators have different mechanical properties such that they apply different forces to the moving element when heated.

33. An implantable medical device comprising a flow control device according to claim 1.

34. A method of controlling an actuation apparatus, the actuation apparatus having a static part and a movable element movable relative to the static part, and an actuator arrangement, the actuator arrangement having first and second actuators connected to the movable element, the method including the step of either:
supplying energy to the first actuator thereby causing the first actuator to exert a force on the movable element and to move relative to the static part in a first direction, or
supplying energy to the second actuator thereby causing the second actuator to exert a force on the movable element and to move the movable element relative to the static part in a second direction which is opposite to said first direction,
wherein energy to cause the movable element to move relative to the static part in one of the first and second directions is supplied via a first energy-receiving region coupled to, or including, both of the first and second actuators,
further wherein the valve is arranged such that the movable element does not move relative to the static part when no energy is supplied to both the first actuator and the second actuator.

35. A method of controlling an actuation apparatus according to claim 34 wherein energy to cause the movable element to move relative to the static part in the other direction is also supplied via the first energy-receiving region; and/or:
wherein the first and second actuators are formed from heat-activated material, the steps of supplying energy including either:
inductively coupling to the first actuator at a first predetermined frequency so as to induce a current flow in the first actuator, or
inductively coupling to the second actuator at a second predetermined frequency,
which is different from said first predetermined frequency, so as to induce a current flow in the second actuator; or
wherein the first and second actuators are formed from heat-activated material, the steps of supplying energy including either:
irradiating a device with radiation at a first predetermined frequency, which radiation is absorbed by the first actuator to a greater extent than it is absorbed by the second actuator, so as to heat the first actuator relative to the second actuator, or
irradiating the device with radiation at a second predetermined frequency, which is different from said first predetermined frequency, and which radiation is absorbed by the second actuator to a greater extent than it is absorbed by the first actuator, so as to heat the second actuator relative to the first actuator; or
wherein the first and second actuators are formed from heat-activated material, the steps of supplying energy including either:
irradiating the device with radiation such that said radiation is incident on the first actuator and is not incident on the second actuator, so as to heat the first actuator relative to the second actuator, or
irradiating the device with radiation such that said radiation is incident on the second actuator and is not incident on the first actuator, so as to heat the second actuator relative to the first actuator.

36-38. (canceled)

39. A method of controlling an actuation apparatus according to claim 34 wherein the first and second actuators are asymmetric such that supply of energy to the flow control device as a whole results in selective actuation of either the first or the second actuator based on one or more of the following characteristics of the supplied energy: the rate at which the energy is supplied; the time period over which the energy is supplied; the total amount of energy supplied; and/or the time-profile of the rate of energy supplied; and optionally:
wherein the first and second actuators have different material properties such that the first actuator has a higher actuation temperature than the second actuator and the steps of supplying energy include:
actuating the first actuator by supplying a first dose of heat energy to the flow control device at a position proximal to the first actuator, the first dose delivering sufficient energy to cause actuation of the first actuator, the duration of the supply of the first dose being sufficiently short to prevent transfer of sufficient energy to the second actuator to cause actuation of the second actuator and thus causing movement of the movable element in the first direction;
actuating the second actuator by supplying a second dose of heat energy to the flow control device at a position proximal to the first actuator, the second dose being of lower power and longer duration than the first dose, such that the second dose is sufficiently long for sufficient heat energy to transfer to the second actuator to cause actuation of the second actuator, but insufficient powerful to cause actuation of the first actuator, and thus causing movement of the movable element in the second direction; or
wherein the first and second actuators have different mechanical properties such that, the second actuator, when actuated, exerts a greater force on the movable element than the first actuator, when actuated, and the steps of supplying energy include:
actuating the first actuator by supplying a first dose of heat energy to the flow control device at a position proximal to the first actuator, the first dose delivering sufficient energy to cause actuation of the first actuator, the duration of the supply of the first dose being sufficiently short to prevent transfer of sufficient energy to the second actuator to cause actuation of the second actuator, and thus causing movement of the movable element in the first direction;
actuating the second actuator by supplying a second dose of heat energy to the flow control device at a position proximal to the first actuator, the second dose being of longer duration than the first dose, such that the second dose is sufficiently long for sufficient heat energy to transfer to the second actuator to cause actuation of the second actuator, and thus causing movement of the movable element in the second direction as a result of the greater force exerted on the movable element by the second actuator compared to the force exerted by the first actuator.

40-41. (canceled)

42. A method of controlling an actuation apparatus according to claim 34 wherein the actuation apparatus is arranged to control the flow rate through a flow control device.

43. An implantable medical device comprising an actuation apparatus according to claim 23.
